(19) United States
(12) Patent Application Publication
(10) Pub. No.: US 2025/0163169 A1
(43) Pub. Date: May 22, 2025

(54) ANTIBODIES FOR PREVENTING THE CLEAVAGE OF CD95L BY METALLOPROTEASES

(71) Applicants: INSTITUT NATIONAL DE LA SANTÉ ET DE LA RECHERCHE MÉDICALE, Paris (FR); CENTRE HOSPITALIER UNIVERSITAIRE DE BORDEAUX, Talence (FR); UNIVERSITÉ DE BORDEAUX, Bordeaux (FR); CENTRE NATIONAL DE LA RECHERCHE SCIENTIFIQUE, Paris (FR); UNIVERSITÉ DE LIMOGES, Limoges (FR); UNIVERSITÉ DE RENNES, RENNES (FR); ÉCOLE NATIONALE SUPÉRIEURE DE CHIMIE DE RENNES (ENSCR), Rennes (FR)

(72) Inventors: Patrick LEGEMBRE, Limoges (FR); Patrick BLANCO, Bordeaux (FR); Mickael JEAN, Rennes (FR)

(21) Appl. No.: 18/839,583

(22) PCT Filed: Feb. 24, 2023
(86) PCT No.: PCT/EP2023/054671

§ 371 Date: Aug. 19, 2024

(30) Foreign Application Priority Data
Feb. 25, 2022 (EP) 22305213.5

(51) Int. Cl. C07K 16/28

(52) U.S. Cl. CPC C07K 16/2875; C07K 2317/24; C07K 2317/76

(57) ABSTRACT

CD95L is a transmembrane glycoprotein that acts through cell contact. After cleavage by metalloproteases, a soluble CD95L (cleaved CD95L or cl-CD95L) is released into the bloodstream. This soluble ligand contributes to aggravate chronic inflammatory disorders such as systemic lupus erythematosus (SLE) and may exert pro-oncogenic functions by promoting the survival of ovarian and breast cancers and chemotherapy resistance of lung cancers. The inventors hypothesized that monoclonal antibodies targeting the Metalloprotease-Cleavage Sites of CD95L (MCSCs) encompassing amino-acid residues 103 to 145 could prevent the access to metalloproteases and inhibit CD95L shedding. Therefore, they generated antibodies covering different epitopes within MCSC and two of them (i.e., 3C7 and 9F5), efficiently abrogated the cleavage of CD95L by MMP2 and MMP3. The results indicate that these MCSC-targeting mAbs can compete with MMPs and represent original and attractive therapeutic options to block the release of the inflammatory s-CD95L.

### FIELD OF THE INVENTION

The present invention is in the field of medicine, in particular immunology and oncology.

### BACKGROUND OF THE INVENTION

CD95L (FasL) belongs to the Tumor Necrosis Factor (TNF) family and is the ligand of the death receptor CD95 (also known as Fas). While CD95 is ubiquitously expressed on healthy cells, CD95L exhibits a restricted expression pattern, mainly detected at the surface of lymphocytes, where it plays a pivotal role in the elimination of infected and transformed cells (Strasser, A.; Jost, P. J.; Nagata, S. The many roles of FAS receptor signaling in the immune system. Immunity. 2009, 30, 180-192). CD95L is a transmembrane glycoprotein (m-CD95L) that acts locally through cell-to-cell contact and after cleavage by metalloproteases such as MMP3 (Matsuno, H.; et al. J Rheumatol. 2001, 28, 22-28.), MMP7 (Vargo-Gogola, T et al. Arch Biochem Biophys. 2002, 408, 155-161), MMP9 (Kiaei, M. et al. Exp Neurol. 2007, 205, 74-81.) or A Disintegrin And Metalloproteinase 10 (ADAM-10) (Kirkin, V et al. The Cell Death Differ. 2007, 14, 1678-1687), a soluble CD95L (named s-CD95L or cleaved-CD95L, cl-CD95L) is released into the bloodstream. This soluble ligand contributes to aggravate inflammation in chronic inflammatory disorders such as systemic lupus erythematosus (SLE) (Tauzin, S. et al. PLoS Biol. 2011, 9, e1001090.) by inducing non-apoptotic signaling pathways such as NF-κB and PI3K and may exert pro-oncogenic functions by promoting the survival of ovarian and liver cancers and chemotherapy resistance of lung cancers. CD95L receptor, designated CD95 or Fas carries an intracellular conserved stretch, the death domain (DD), which serves as a docking platform to trigger cell death. Binding of membrane-bound hexameric CD95L to CD95 leads to the recruitment of the adaptor protein FADD (Fas Associated Death Domain) through homotypic interactions via their respective DD (Holler, N. et al., Mol Cell Biol. 2003, 23, 1428-1440). FADD in turn aggregates the initiator caspase-8 and caspase-10. The CD95/FADD/caspase complex is called death-inducing signalling complex (DISC) and leads to the elimination of cancer cells through an apoptotic mechanism (Kischkel, F. C. et al. Embo J. 1995, 14, 5579-5588). By contrast, homotrimeric cl-CD95L fails to induce DISC formation, but instead triggers the formation of a non-apoptotic complex termed motility-inducing signaling complex (MISC) implementing a Ca²⁺ response (Tauzin, S. et al. PLoS Biol. 2011, 9, e1001090.). Recent data highlighted that cl-CD95L induces a calcium response by inducing the direct interaction of CD95 with PLCγ1 (Poissonnier, A. et al. Immunity. 2016, 45, 209-223). Indeed, in presence of cl-CD95L, the juxtamembrane region of CD95, called calcium-inducing domain (CID), recruits PLCγ1 to induce endothelial transmigration of Th17 cells in SLE (Poissonnier, A. et al. Immunity. 2016, 45, 209-223). Moreover, a peptide consisting of the CID conjugated to the cell-penetrating domain of TAT, namely TAT-CID, binds PLCγ1 and prevents its recruitment to CD95. Strikingly, repeated injections of TAT-CID in lupus-prone mice (MRL^{Lpr/+}) block endothelial transmigration of Th17 cells and alleviate clinical symptoms (Poissonnier, A. et al. Immunity. 2016, 45, 209-223).

### SUMMARY OF THE INVENTION

The present invention is defined by the claims. In particular, the present invention relates to antibodies for preventing the cleavage of CD95L by metalloproteases.

### DETAILED DESCRIPTION OF THE INVENTION

CD95L is a transmembrane glycoprotein that acts locally through cell-cell contact. After cleavage by metalloproteases, a soluble CD95L (cleaved CD95L or cl-CD95L) is released into the bloodstream. This soluble ligand contributes to aggravate inflammation in chronic inflammatory disorders such as systemic lupus erythematosus (SLE) and may exert pro-oncogenic functions by promoting the survival of ovarian and liver cancers and chemotherapy resistance of lung cancers. The inventors hypothesized that monoclonal antibodies targeting the Metalloprotease-Cleavage Sites of CD95L (MCSCs), also designated stalk region, encompassing amino-acid residues 103 to 145 could prevent the access to metalloproteases and thereby, inhibit CD95L shedding. Therefore, they generated antibodies covering different epitopes within MCSC and two of them (i.e., 3C7 and 9F5), efficiently abrogated the cleavage of CD95L by MMP2 and MMP3. The results indicate that these MCSC-targeting mAbs can compete with MMPs and represent original and attractive therapeutic options to block the release of the inflammatory s-CD95L.

Accordingly, a first object of the present invention relates to an antibody which binds to the extracellular domain of CD95L, wherein said antibody prevents the cleavage of CD95L by at least one metalloprotease and wherein the epitope of said antibody comprises at least one amino acid residue in the amino acid sequence ranging from the amino acid residue at position 110 to the amino acid residue at position 130 in SEQ ID NO:1.

The present invention relates also to an antibody which binds to the extracellular domain of CD95L, wherein said antibody prevents the cleavage of CD95L by at least one metalloprotease and wherein the epitope of said antibody comprises at least one amino acid residue in the amino acid sequence ranging from the amino acid residue at position 103 to the amino acid residue at position 130 in SEQ ID NO:1.

As used herein, the term “CD95L” has its general meaning in the art and refers to the cognate ligand of CD95 that is a transmembrane protein. The term is also known as APT1LG1, FASL, or TNFSF6. An exemplary amino acid sequence for CD95L is represented by SEQ ID NO:1. The extracellular domain typically consists of the amino acid sequence that ranges from the amino acid residue at position 103 to the amino acid residue at position 281 in SEQ ID NO:1.

SEQ ID NO: 1
>sp|P48023|TNFL6_HUMAN Tumor necrosis factor ligand
superfamily member 6 OS = Homo sapiens OX = 9606 GN = FASLG PE = 1 SV = 1. The
extracellular domain is underlined in the sequence. The epitope
location is indicated in bold.
MQQPFNYPYPQIYWVDSSASSPWAPPGTVLPCPTSVPRRPGQRRPPPPPPPPPLPPPPPPPPLPPLPLP

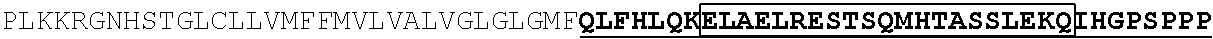

EKKELRKVAHLTGKSNSRSMPLEWEDTYGIVLLSGVKYKKGGLVINETGLYFVYSKVYFRGQSCNNLPL
SHKVYMRNSKYPQDLVMMEGKMMSYCTTGOMWARSSYLGAVENLTSADHLYVNVSELSLVNFEESQTFF
GLYKL

As used herein the term “soluble CD95L” has its general meaning in the art and refers to the soluble ligand produced by the cleavage of the transmembrane CD95L by a metalloprotease. The term “serum CD95L”, “soluble CD95L”, “metalloprotease-cleaved CD95L” and “cl-CD95L” have the same meaning along the specification.

As used herein, the term “metalloprotease” or “MMP” has its general meaning in the art and refers to an enzyme that uses a metal-activated water, e.g., zinc-activated water, to catalyse the hydrolytic degradation of proteins or polypeptides to smaller amino acid polymers. Examples of MMP according to the present invention include MMP-2, MMP-3 MMP-7, MMP12 and MMP13. In particular, the monoclonal antibody of the present invention prevents the cleavage of CD95L by MMP-2 and/or MMP-3.

As used herein the term “antibody” or “immunoglobulin” has the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four (α, δ, γ) to five (μ, ε) domains, a variable domain (VH) and three to four constant domains (CH1, CH2, CH3 and CH4 collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. According to the invention, the amino acid residues in the variable domain, complementarity determining regions (CDRs) and framework regions (FR) of the antibody of the present invention are identified using the Immunogenetics (IMGT) database (http://imgt.cines.fr). Lefranc et al. (2003) Dev Comp Immunol. 27(1):55-77. The IM4GT database was developed using sequence information for immunoglobulins (IgGs), T-cell receptors (TcR) and Major Histocompatibility Complex (MHC) molecules and unifies numbering across antibody lambda and kappa light chains, heavy chains and T-cell receptor chains and avoids the use of insertion codes for all but uncommonly long insertions. IMGT also takes into account and combines the definition of the framework (FR) and complementarity determining regions (CDR) from Kabat et al., the characterization of the hypervariable loops from Chothia et al., as well as structural data from X-ray diffraction studies.

As used herein, the term “specificity” refers to the ability of an antibody to detectably bind target molecule (e.g. an epitope presented on an antigen) while having relatively little detectable reactivity with other target molecules. Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

The term “affinity”, as used herein, means the strength of the binding of an antibody to a target molecule (e.g. an epitope). The affinity of a binding protein is given by the dissociation constant Kd. For an antibody said Kd is defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of a binding protein can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of binding protein is the use of Biacore instruments.

The term “binding” as used herein refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined target molecule (e.g. an antigen or epitope) typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less.

As used herein, the term “epitope” refers to a specific arrangement of amino acids located on a protein or proteins to which an antibody binds. Epitopes often consist of a chemically active surface grouping of molecules such as amino acids or sugar side chains, and have specific three-dimensional structural characteristics as well as specific charge characteristics. Epitopes can be linear or conformational, i.e., involving two or more sequences of amino acids in various regions of the antigen that may not necessarily be contiguous.

In some embodiments, the antibody of the invention binds to an epitope comprising at least one amino acid residue in the amino acid sequence ranging from the amino acid residue at position 110 to the amino acid residue at position 129 in SEQ ID NO:1.

In some embodiments, the antibody of the invention binds to an epitope comprising or consisting of 1, 2, 3, 4, 5, 6, 7, 8, 9, 10, 11, 12, 13, 14, 15, 16, 17, 18 19, 20 or 21 amino acid residues from amino acid residues 110 to 130 of SEQ ID NO: 1.

In some embodiments, the antibody of the invention binds to an epitope comprising or consisting of 1, 2, 3, 4, 5, 6, 7, 8, 9, 10, 11, 12, 13, 14, 15, 16, 17, 18 19, 20, 21, 22, 23, 24, 25, 26 or 27 amino acid residues from amino acid residues 103 to 130 of SEQ ID NO: 1.

In some embodiments, the antibody of the invention binds to an epitope comprising or consisting of the amino acid sequence ranging from the amino acid residue at position 110, 111, 112, 113, 114, 115, 116, 117, 118, 119, 120, 121, 122 or 123 to the amino acid residue at position 129 or 130 of SEQ ID NO:1.

In some embodiments, the antibody of the invention binds to an epitope comprising or consisting of the amino acid sequence ranging from the amino acid residue at position 122 to the amino acid residue at position 129 of SEQ ID NO:1. In other words, in some embodiments, the antibody of the invention binds to an epitope comprising or consisting of sequences set forth as HTASSLEK (SEQ ID NO:24).

In some embodiments, the antibody of the invention binds to a linear epitope.

In some embodiments, the antibody of the invention binds to a conformational epitope.

In some embodiments, the antibody of the invention binds to a linear or conformational epitope comprising or consisting in 1, 2, 3, 4, 5, 6, 7, 8, 9, 10, 11, 12, 13, 14, 15, 16, 17, 18, 19, 20 or 21 amino acid residues from amino acid residues 110 to 130 of SEQ ID NO: 1.

In some embodiments, the antibody of the invention binds to a linear or conformational epitope comprising or consisting of 1, 2, 3, 4, 5, 6, 7, 8, 9, 10, 11, 12, 13, 14, 15, 16, 17, 18 19, 20, 21, 22, 23, 24, 25, 26 or 27 amino acid residues from amino acid residues 103 to 130 of SEQ ID NO: 1.

In some embodiments, the antibody of the invention binds to a linear or conformational epitope comprising or consisting of the amino acid sequence ranging from the amino acid residue at position 110, 111, 112, 113, 114, 115, 116, 117, 118, 119, 120, 121, 122 or 123 to the amino acid residue at position 129 or 130 of SEQ ID NO:1.

In some embodiments, the antibody of the invention binds to a linear or conformational epitope comprising or consisting of the amino acid sequence ranging from the amino acid residue at position 122 to the amino acid residue at position 129 of SEQ ID NO: 1.

In some embodiments, the antibody of the invention binds to a peptide comprising or consisting of sequences set forth as HTASSLEK (SEQ ID NO:24).

In some embodiments, the antibody of the invention binds to a peptide comprising or consisting of sequences set forth as LAELRESTSQMHTASSLEK (SEQ ID NO:22).

In some embodiments, the antibody of the invention binds to a peptide comprising or consisting of sequences set forth as LAELRESTSQMHTASSLEK (SEQ ID NO:22) and/or HTASSLEKQIGHPSP (SEQ ID NO:23).

In some embodiments, the antibody of the present invention is a monoclonal antibody. The terms “monoclonal antibody”, “monoclonal Ab”, “monoclonal antibody composition”, “mAb”, or the like, as used herein refer to a preparation of antibody molecules of single molecular composition. A monoclonal antibody is obtained from a population of substantially homogeneous antibodies, i.e., the individual antibodies comprised in the population are identical except for possible naturally occurring mutations that may be present in minor amounts. A monoclonal antibody composition displays a single binding specificity and affinity for a particular epitope. For instance, monoclonal antibodies may be generated using the method of Kohler and Milstein (Nature, 256:495, 1975). To prepare monoclonal antibodies useful in the invention, a mouse or other appropriate host animal can be immunized at suitable intervals (e.g., twice-weekly, weekly, twice-monthly or monthly) with the appropriate antigenic forms (i.e., CD95L polypeptides). The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes. Monoclonal antibodies useful in the invention can also be prepared from non-immunized animals or humans. However, the modifier “monoclonal” is not to be construed as requiring production of the antibody by any particular method. For example, a monoclonal antibody may also be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). A “monoclonal antibody” may also be isolated from phage antibody libraries using the techniques described in Clackson et al., Nature, 352:624-628 (1991) and Marks et al., J. Mol. Biol., 222:581-597 (1991), for example.

In some embodiments, the antibody of the present invention is a chimeric antibody. As used herein, the term “chimeric antibody” refers to an antibody which comprises a VH domain and a VL domain of a non-human antibody, and a CH domain and a CL domain of a human antibody.

In some embodiments, the antibody of the present invention is a humanized antibody. The term “humanized antibody” refers to an antibody having variable region framework and constant regions from a human antibody but retains the CDRs of a previous non-human antibody. In some embodiments, a humanized antibody contains minimal sequence derived from non-human immunoglobulin. For the most part, humanized antibodies and antibody fragments thereof may be human immunoglobulins (recipient antibody or antibody fragment) in which residues from a complementary-determining region (CDR) of the recipient are replaced by residues from a CDR of non-human species (donor antibody) such as mouse, rat or rabbit having the desired specificity, affinity, and capacity.

In some embodiments, the antibody of the present invention is a human antibody. As used herein the term “human monoclonal antibody”, is intended to include antibodies having variable and constant regions derived from human immunoglobulin sequences. The human antibodies of the present invention may include amino acid residues not encoded by human immunoglobulin sequences (e.g., mutations introduced by random or site-specific mutagenesis in vitro or by somatic mutation in vivo). More specifically, the term “human monoclonal antibody”, as used herein, is not intended to include antibodies in which CDR sequences derived from the germline of another mammalian species, such as a mouse, have been grafted onto human framework sequences.

In some embodiments, the antibody of the present invention an antibody fragment. As used herein, the term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, single chain antibody molecules, in particular scFv antibody fragments, disulfide-linked Fvs (sdFv), a Fd fragment consisting of the VH and CH1 domains, linear antibodies, single domain antibodies such as, for example, sdAb (either VL or VH), camelid VHH domains, multi-specific antibodies formed from antibody fragments such as, for example, a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (see U.S. Pat. No. 6,703,199, which describes fibronectin polypeptide minibodies). Papain digestion of antibodies produces two identical antigen-binding fragments, called “Fab” fragments, and a residual “Fc” fragment, a designation reflecting the ability to crystallize readily.

Fragments and derivatives of antibodies of this invention (which are encompassed by the term “antibody” as used in this application, unless otherwise stated or clearly contradicted by context), can be produced by techniques that are known in the art. “Fragments” comprise a portion of the intact antibody, generally the antigen binding site or variable region. Examples of antibody fragments include Fab, Fab′, Fab′-SH, F(ab′)2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Fragments of the present antibodies can be obtained using standard methods.

For instance, Fab or F(ab′)₂ fragments may be produced by protease digestion of the isolated antibodies, according to conventional techniques. It will be appreciated that immunoreactive fragments can be modified using known methods, for example to slow clearance in vivo and obtain a more desirable pharmacokinetic profile the fragment may be modified with polyethylene glycol (PEG). Methods for coupling and site-specifically conjugating PEG to a Fab′ fragment are described in, for example, Leong et al., Cytokines 16 (3): 106-119 (2001) and Delgado et al., Br. J. Cancer 5 73 (2): 175-182 (1996), the disclosures of which are incorporated herein by reference.

In some embodiments, the antibody is a nanobody. As used herein the term “nanobody” has its general meaning in the art and refers to an antibody-derived therapeutic protein that contains the unique structural and functional properties of naturally-occurring heavy chain antibodies. These heavy chain antibodies contain a single variable domain (VHH) and two constant domains (CH2 and CH3). As used herein, the term “derived” indicates a relationship between a first and a second molecule. It generally refers to structural similarity between the first molecule and the second molecule and does not connote or include a process or source limitation on a first molecule that is derived from a second molecule.

In some embodiments, the antibody of the invention comprises a light chain comprising at least one or at least two of the following CDRs:

(SEQ ID NO: 2)
L-CDR1: QSLLSSR-X₈-QK-X₁₁-Y
wherein X₈ is T or N and X₁₁ is H or T
(SEQ ID NO: 3)
L-CDR2: FTS
(SEQ ID NO: 4)
L-CDR3: X₁-QHYNTPWT
wherein X₁ is K or Q

In some embodiments, the antibody of the invention comprises a heavy chain comprising at least one or at least two of the following CDRs:

(SEQ ID NO: 5)
H-CDR1: GYTFT-X₆-YW
wherein X₆ is S or T
(SEQ ID NO: 6)
H-CDR2: INP-X₄-X₅-GYT
wherein X₄ is N or R and X₅ is T or N
(SEQ ID NO: 7)
H-CDR3: AVDLGY

In some embodiment, the antibody of the invention comprises a light chain comprising at least one of the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and/or a heavy chain comprising at least one of the following CDR i) the VH-CDR1 as set forth in SEQ ID NO: 10 (GYTFTSYW), ii) the VH-CDR2 as set forth in SEQ ID NO:11 (INPNTGYT) and iii) the VH-CDR3 as set forth in SEQ ID NO:7 (AVDLGY).

In some embodiment, the antibody of the invention comprises a light chain comprising at least one of the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO: 13 (QQHYNTPWT) and/or a heavy chain comprising at least one of the following CDR i) the VH-CDR1 as set forth in SEQ ID NO: 14 (GYTFTTYW), ii) the VH-CDR2 as set forth in SEQ ID NO:15 (INPRNGYT) and iii) the VH-CDR3 as set forth in SEQ ID NO:7 (AVDLGY).

In some embodiment, the antibody of the invention comprises a light chain comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and a heavy chain comprising the following CDR i) the VH-CDR1 as set forth in SEQ ID NO: 10 (GYTFTSYW), ii) the VH-CDR2 as set forth in SEQ ID NO:11 (INPNTGYT) and iii) the VH-CDR3 as set forth in SEQ ID NO:7 (AVDLGY) (“3C7 antibody”).

In some embodiment, the antibody of the invention comprises a light chain comprising the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and a heavy chain comprising the following CDR i) the VH-CDR1 as set forth in SEQ ID NO: 14 (GYTFTTYW), ii) the VH-CDR2 as set forth in SEQ ID NO:15 (INPRNGYT) and iii) the VH-CDR3 as set forth in SEQ ID NO:7 (AVDLGY) (“9F5 antibody”).

In some embodiments, the antibody of the present invention comprises a light chain having 70% of identity with SEQ ID NO: 16 or SEQ ID NO: 18 and/or a heavy chain having 70% of identity with SEQ ID NO: 17 or SEQ ID NO:19.

In some embodiments, the antibody of the present invention comprises a light chain having 70% of identity with SEQ ID NO: 16 and/or a heavy chain having 70% of identity with SEQ ID NO: 17 (“3C7 antibody”).

In some embodiments, the antibody of the present invention comprises a light chain having 70% of identity with SEQ ID NO: 16 and comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT), and/or a heavy chain having 70% of identity with SEQ ID NO: 17 and comprising the following CDR i) the VH-CDR1 as set forth in SEQ ID NO: 10 (GYTFTSYW), ii) the VH-CDR2 as set forth in SEQ ID NO:11 (INPNTGYT) and iii) the VH-CDR3 as set forth in SEQ ID NO:7 (AVDLGY) (“3C7 antibody”).

In some embodiments, the antibody of the present invention comprises a light chain having an amino acid sequence as set forth in SEQ ID NO: 16 and/or a heavy chain having an amino acid sequence as set forth in SEQ ID NO: 17 (“3C7 antibody”).

In some embodiments, the antibody of the present invention comprises a light chain having 70% of identity with SEQ ID NO:18 and/or a heavy chain having 70% of identity with SEQ SEQ ID NO:19 (“9F5 antibody”).

In some embodiments, the antibody of the present invention comprises a light chain having 70% of identity with SEQ ID NO:18 and comprising the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT), and/or a heavy chain having 70% of identity with SEQ SEQ ID NO:19 and comprising the following CDR i) the VH-CDR1 as set forth in SEQ ID NO:14 (GYTFTTYW), ii) the VH-CDR2 as set forth in SEQ ID NO:15 (INPRNGYT) and iii) the VH-CDR3 as set forth in SEQ ID NO:7 (AVDLGY) (“9F5 antibody”).

In some embodiments, the antibody of the present invention comprises a light chain having an amino acid sequence as set forth in SEQ ID NO: 18 and/or a heavy chain having an amino acid sequence as set forth in SEQ ID NO: 19 (“9F5 antibody”).

SEQ ID NO: 16: light chain of the antibody 3C7-3H1 FR1-CDR1-FR2-
CDR2-FR3-CDR3-FR4
DIVMTQSPSSLAMSVGQKVTMSCKSS QSLLSSRTQKHY LAWYQQKPGQSPKLLVY
FTS TRESGVPDRFIGSGSGTDFTLTISSVQAEDLALYYC KQHYNTPWT FGGGTKLEIK
SEQ ID NO: 17: heavy chain of the antibody 3C7-3H1 FR1-CDR1-FR2-
CDR2-FR3-CDR3-FR4
QVQLQQSGAELAKPGASVKMSCKTS GYTFTSYW MHWVKQRPGQGLEWIGY INPNTGYT
EYIQRFRDKATLTADKSSGTAYMQLSSLTSEDSAVYYC AVDLGY WGQGTLVTVSA
SEQ ID NO: 18: light chain of the antibody 9F5-3E7 FR1-CDR1-FR2-
CDR2-FR3-CDR3-FR4
DIVMTQSPSSLAMSVGQKVTMNCKSS QSLLSSRNQKTY LAWYQQKPGQSPKLLVY FTS
TRASGVPDRFIGSGGTDFTLTISSVQAEDLALYYC QQHYNTPWT FGGGTKLEIK.
SEQ ID NO: 19: heavy chain of the antibody 9F5-3E7 FR1-CDR1-FR2-
CDR2-FR3-CDR3-FR4
QVQLQQSGAELAKPGASVKMSCKAS GYTFTTYW MHWVKQRPGQDLEWIGY INPRNGYT
EYNQNFKDKATLTADKSSTTAFMQLSSLTSEDSALYYC AVDLGY WGQGTLVTVSA

According to the invention, a first amino acid sequence having at least 70% of identity with a second amino acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0063, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the heavy chain and/or the light chain of the antibody of the invention comprises conservative sequence modifications. The term “conservative sequence modifications” refers to amino acid modifications that do not significantly affect or alter the biologic function of the protein containing the amino acid sequence. Such conservative modifications include amino acid substitutions, additions and deletions. Modifications can be introduced into a protein by standard techniques known in the art, such as site-directed mutagenesis and PCR-mediated mutagenesis. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine. Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Other families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an antibody of the invention can be replaced with other amino acid residues from the same side chain family and the altered antibody can be tested for binding to CD95L.

In some embodiments, the monoclonal antibody of the present invention cross-competes for binding to the CD95L isoform with the monoclonal antibody comprising a light chain comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and a heavy chain comprising the following CDR i) the VH-CDR1 as set forth in SEQ ID NO:10 (GYTFTSYW), ii) the VH-CDR2 as set forth in SEQ ID NO:11 (INPNTGYT) and iii) the VH-CDR3 as set forth in SEQ ID NO:7 (AVDLGY).

In some embodiments, the monoclonal antibody of the present invention cross-competes for binding to the CD95L isoform with the monoclonal antibody comprising a light chain comprising the following CDR: i) the VL-CDR1 as set forth in SEQ ID NO: 12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and heavy chain comprising the following CDR i) the VH-CDR1 as set forth in SEQ ID NO: 14 (GYTFTTYW), ii) the VH-CDR2 as set forth in SEQ ID NO:15 (INPRNGYT) and iii) the VH-CDR3 as set forth in SEQ ID NO:7 (AVDLGY).

As used herein, the term “cross-competes” refers to monoclonal antibodies which share the ability to bind to a specific region of an antigen. In the present disclosure the monoclonal antibody that “cross-competes” has the ability to interfere with the binding of another monoclonal antibody for the antigen in a standard competitive binding assay. Such a monoclonal antibody may, according to non-limiting theory, bind to the same or a related or nearby (e.g., a structurally similar or spatially proximal) epitope as the antibody with which it competes. Cross-competition is present if antibody A reduces binding of antibody B at least by 60%, specifically at least by 70% and more specifically at least by 80% and vice versa in comparison to the positive control which lacks one of said antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

According to the present invention, the cross-competing antibody as above described retain the activity of the monoclonal antibody which comprises a light chain comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and a heavy chain comprising the following CDR i) the VH-CDR1 as set forth in SEQ ID NO: 10 (GYTFTSYW), ii) the VH-CDR2 as set forth in SEQ ID NO:11 (INPNTGYT) and iii) the VH-CDR3 as set forth in SEQ ID NO:7 (AVDLGY).

According to the present invention, the cross-competing antibody as above described retain the activity of the monoclonal antibody which comprises a light chain comprising the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and heavy chain comprising the following CDR i) the VH-CDR1 as set forth in SEQ ID NO:14 (GYTFTTYW), ii) the VH-CDR2 as set forth in SEQ ID NO:15 (INPRNGYT) and iii) the VH-CDR3 as set forth in SEQ ID NO:7 (AVDLGY).

Any assay well known in the art would be suitable for identifying whether the cross-competing antibody retains the desired activity. For instance, the assay described in EXAMPLE that consist in determining the ability of preventing the cleavage of CD95L by metalloproteases such as MMP-2 or MP-3 would be suitable for determining whether the antibody retains said ability.

The antibody of the present invention is produced by any technique known in the art, such as, without limitation, any chemical, biological, genetic or enzymatic technique, either alone or in combination. Typically, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, California) and following the manufacturer's instructions. Alternatively, antibodies of the present invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

Accordingly, a further object of the invention relates to a nucleic acid molecule encoding an antibody according to the invention. More particularly the nucleic acid molecule encodes a heavy chain and/or a light chain of an antibody of the present invention.

As used herein, the term “encoding” refers to the inherent property of specific sequences of nucleotides in a polynucleotide, such as, for example, a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase “nucleotide sequence that encodes a protein or a RNA” may also include introns to the extent that the nucleotide sequence encoding the protein may in some version contain an intron(s).

Typically, said nucleic acid is a DNA or RNA molecule, which may be included in any suitable vector, such as a plasmid, cosmid, episome, artificial chromosome, phage or a viral vector. As used herein, the terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. So, a further object of the invention relates to a vector comprising a nucleic acid of the invention. Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40, LTR promoter and enhancer of Moloney mouse leukemia virus, promoter and enhancer of immunoglobulin H chain and the like. Any expression vector for animal cell can be used, so long as a gene encoding the human antibody C region can be inserted and expressed. Examples of suitable vectors include pAGE107, pAGE103, pHSG274, pKCR, pSG1 beta d2-4 and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The term “promoter/regulatory sequence” refers to a nucleic acid sequence (such as, for example, a DNA sequence) recognized by the synthetic machinery of the cell, or introduced synthetic machinery, required to initiate the specific transcription of a polynucleotide sequence, thereby allowing the expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

A further object of the present invention relates to a host cell which has been transfected, infected or transformed by a nucleic acid and/or a vector according to the invention.

In particular embodiment, the host cell comprises a nucleic acid and/or a vector according to the invention. As used herein, the term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

The nucleic acids of the invention may be used to produce an antibody of the present invention in a suitable expression system. The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the present invention.

Examples of vectors include all those known in the art, including, without limitation, cosmids, plasmids (e.g., naked or contained in liposomes) and viruses (e.g., lentiviruses, retroviruses, adenoviruses, and adeno-associated viruses) that incorporate the recombinant polynucleotide.

Antibodies of the present invention are suitably separated from the culture medium by conventional immunoglobulin purification procedures such as, for example, protein A-Sepharose, hydroxylapatite chromatography, gel electrophoresis, dialysis, or affinity chromatography.

In some embodiments, the antibody (preferably the monoclonal antibody) of the present invention does not comprise a Fc region that mediates antibody-dependent cell-mediated cytotoxicity and thus does not comprise an Fc portion that induces antibody dependent cellular cytotoxicity (ADCC). In some embodiments, the antibody (preferably the monoclonal antibody) of the present invention does not comprise an Fc region that induces complement dependent cytotoxicity (CDC) or antibody-dependent phagocytosis. In some embodiments the antibody (preferably the monoclonal antibody) of the present invention does not lead, directly or indirectly, to the depletion of cells expressing CD95L polypeptides (e.g., do not lead to a 10%, 20%, 50%, 60% or greater elimination or decrease in number of CD95L⁺ Cells). In some embodiments, the antibody (preferably the monoclonal antibody) of the present invention does not comprise an Fc domain capable of substantially binding to a FcγRIIIA (CD16) polypeptide. In some embodiments, the antibody (preferably the monoclonal antibody) of the present invention lacks an Fc domain (e.g., lacks a CH2 and/or CH3 domain) or comprises an Fc domain of IgG2 or IgG4 isotype. In some embodiments, the antibody (preferably the monoclonal antibody) of the present invention comprises an Fc domain (e.g. of IgG1) with an altered glycosylation profile, resulting in the absence of ADCC activity of the antibody. In some embodiments, the antibody (preferably the monoclonal antibody) of the present invention consists of or comprises a Fab, Fab′, Fab′-SH, F(ab′)₂, Fv, a diabody, single-chain antibody fragment, or a multispecific antibody comprising multiple different antibody fragments. In some embodiments, the antibody (preferably the monoclonal antibody) of the present invention is not linked to a toxic moiety. In some embodiments, one or more amino acids selected from amino acid residues can be replaced with a different amino acid residue such that the antibody has altered C2q binding and/or reduced or abolished CDC. This approach is described in further detail in U.S. Pat. No. 6,194,551 by ldusogie et al.

As used herein, the term “Fc region” includes the polypeptides comprising the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

The terms “Fc receptor” or “FcR” are used to describe a receptor that binds to the Fc region of an antibody. The primary cells for mediating ADCC, Cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term “effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

Accordingly a further object of the present invention relates to the antibody of the present invention for use as a drug. More specifically, the present invention provides a method of therapy in a patient in need thereof, comprising administering to the patient a therapeutically effective amount of an antibody of the present invention.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Also encompassed by “treatment” is a reduction of pathological consequence of cancer. The methods of the present invention contemplate any one or more of these aspects of treatment. In some embodiments, the terms “treating” or “treatment” refers to both therapeutic treatment and prophylactic or preventative measures; wherein the object is to prevent or slow down (lessen) the targeted disease. Therefore, in some embodiments, those in need of treatment may include those already with the disorder as well as those prone to have the disorder or those in whom the disorder is to be prevented.

In some embodiments, the antibody of the present invention is particularly the treatment of cancer in a subject in need thereof. As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may be treated by methods and compositions of the present invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject suffers from a cancer selected from the group consisting of breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

In some embodiments, the antibody of the present invention is particularly for the treatment of triple negative breast cancer. As used herein the expression “triple negative breast cancer” has its general meaning in the art and means that said breast cancer lacks or expresses low levels of receptors for the hormones estrogen (ER-negative) and progesterone (PR-negative), and for the protein HER2.

In some embodiments, the antibody of the present invention is particularly for the prevention of metastases (e.g. in a subject suffering from a triple negative breast cancer).

In some embodiments, the antibody of the present invention is particularly for enhancing therapeutic efficacy of cancer treatment in a subject in need thereof. In some embodiments, the method of the present invention comprises administering the subject with a therapeutically effective amount of an antibody of the present invention sequentially or concomitantly with one or more therapeutic active agent such as chemotherapeutic or radiotherapeutic agents. Examples of chemotherapeutics include but are not limited to fludarabine, gemcitabine, capecitabine, methotrexate, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbazine, epipodophyllotoxins such as etoposide and teniposide, camptothecins such as irinotecan and topotecan, bleomycin, doxorubicin, idarubicin, dactinomycin, plicamycin, mitoxantrone, L-asparaginase, doxorubicin, epirubicin, 5-fluorouracil and 5-fluorouracil combined with leucovorin, taxanes such as docetaxel and paclitaxel, levamisole, estramustine, nitrogen mustards, nitrosoureas such as carmustine and lomustine, vinca alkaloids such as vinblastine, vincristine, vindesine and vinorelbine, imatinib mesylate, hexamethylmelamine, kinase inhibitors, phosphatase inhibitors, ATPase inhibitors, tyrphostins, protease inhibitors, inhibitors herbimycin A, genistein, erbstatin, and lavendustin A. In some embodiments, additional therapeutic active agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxins, hormonal therapies, retinoids, photosensitizers or photodynamic therapies, angiogenesis inhibitors, antimitotic agents, isoprenylation inhibitors, cell cycle inhibitors, actinomycin, bleomycin, anthracyclines, MDR inhibitors and Ca2+ ATPase inhibitors. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In some embodiments, the antibody of the present invention is particularly for the treatment of an autoimmune inflammatory disease. In some embodiments, the autoimmune inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal gammopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis (e.g. chronic pancreatitis), polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, the antibody of the present invention is particularly suitable for the treatment of systemic lupus erythematosus.

A further object of the present invention relates to a composition comprising, consisting of or consisting essentially of an antibody of the present invention.

As used herein, “consisting essentially of”, with reference to a composition, means that the at least one antibody of the invention as described hereinabove is the only one therapeutic agent or agent with a biologic activity within said composition.

In some embodiments, the composition of the invention is a pharmaceutical composition and further comprises a pharmaceutically acceptable carrier.

The term “pharmaceutically acceptable carrier” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g., 1 ml for intramuscular), and between about 1 ng to about 100 mg, e.g., about 50 ng to about 30 mg or more preferably, about 5 mg to about 25 mg, of an antibody of the invention.

The invention will be further illustrated by the following figures and examples. However, these examples and figures should not be interpreted in any way as limiting the scope of the present invention.

### FIGURES

FIG. 1. Mapping of the cleavage sites and metalloproteases involved in CD95L. A. In the literature, full length CD95L has been reported to be cleaved at different sites. Arrows represent putative cleavage sites reported in the literature. B. A 43-mer depicted above the histograms and covering the stalk region of CD95L was incubated with indicated MMPs and ADAMs at the same concentration. The cleavage efficiency of 43-mer was assessed by measuring its degradation using LC-MS/MS. A percentage of degradation deduced from the decrease of peak area corresponding to the starting 43-mer was attributed to each single protease. C. From the same experiments, the privileged cleavage sites (¹¹³EL¹¹⁴ for MMPs and ¹²⁹KQ¹³⁰ for ADAM) were determined from MS/MS sequencing of the resulting cleavage products.

FIG. 2. Monoclonal antibodies targeting the Metalloprotease-Cleavage Sites of CD95L (MCSCs). A. Left panel: The extracellular region of CD95L encompassing amino acid residues 102 to 281 can be cleaved by metalloproteases at different sites. Arrows represent putative cleavage site in CD95L sequence. Right panel: Using three peptides (peptides 1, 2 and 3) covering most of the MCSCs of CD95L, we have generated monoclonal antibodies. Indicated peptides were injected in mice to generate monoclonal antibodies and three different hybridoma were selected regarding their ability to bind CD95L. According to their affinity for the indicated peptides (assessed using ELISA), the 3C7 epitope should cover the HTASSLEK sequence (SEQ ID NO:24). B. Recombinant Ig-CD95L (100 ng) was pre-incubated for 15 min with indicated antibodies (100 pg/mL) and then treated for 1h with the indicated MMPs. The metalloprotease-cleaved fragment of Ig-CD95L (CD95L devoid of its released Ig domain of gp190) was monitored by immunoblotting. C. CD95L-expressing leukemic T cell 1A12 was incubated for 24 h with indicated antibodies (10 pg/mL) or the pan-MMP inhibitor GM6001 (10 μM) and the expression level of membrane-bound CD95L was assessed by flow cytometry. D. Supernatants were harvested from cells treated in C and the metalloprotease-cleaved CD95L was dosed by ELISA (Diaclone). The concentrations of s-CD95L are significantly reduced in 1A12 cells treated for 24 hours with GM6001 or 3C7 as compared to isotype (anti-myc 9E10 mAb)-treated control cells.

FIG. 3. Antibody targeting the Metalloprotease-Cleavage Sites of CD95L (MCSCs) blocks the non-apoptotic functions of CD95L without affecting its apoptotic ones. A. WR19L (CD95L−) or 1A12 (CD95L+) cells were placed in the lower chamber of a Boyden chamber assay in the presence of the indicated mAbs (10 pg/mL). Triple negative breast cancer cell MDA-MB-231 was added to the upper part of the chamber and cell migration due to the gradient of metalloprotease-cleaved CD95L was evaluated after 24 hours by fixing and staining (Giemsa) migrating cells covering the lower side of the porous membrane. Left panel: Pictures of the Giemsa-stained MDA-MB-231 cells that migrated to the lower side of the membrane in the presence of WR19L (CD95L−) or 1A12 (CD95L+) cells and with isotypic (9E10) or anti-MCSC (3C7) monoclonal antibody. Right panel: Giemsa-stained MDA-MB-231 cells were lysed and absorbance was measured at a wavelength of 560 nm. Values represent the means and SEM of three independently performed experiments. **p<0.01 as calculated using two-tailed Mann-Whitney test. B. Upper panel: Jurkat cells are sensitive to the CD95-mediated apoptotic signal. Jurkat cells were stained with DIOC₆ (measure of the mitochondrial membrane potential) and then incubated for 4 hours at the indicated ratio with 1A12 (CD95L+) or WR19L(CD95L−) cells. Cell death was monitored by measuring the drop of mitochondrial transmembrane potential (Δψm). Lower panel: 1A12 cells were pre-treated for 24 hours with 10 μg/mL of the indicated mAbs, and then washed. Cells were next incubated for 4 hours at the indicated ratio with Jurkat cells stained with DIOC₆. Cell death was monitored by measuring the drop of mitochondrial transmembrane potential (Δψm).

### EXAMPLE

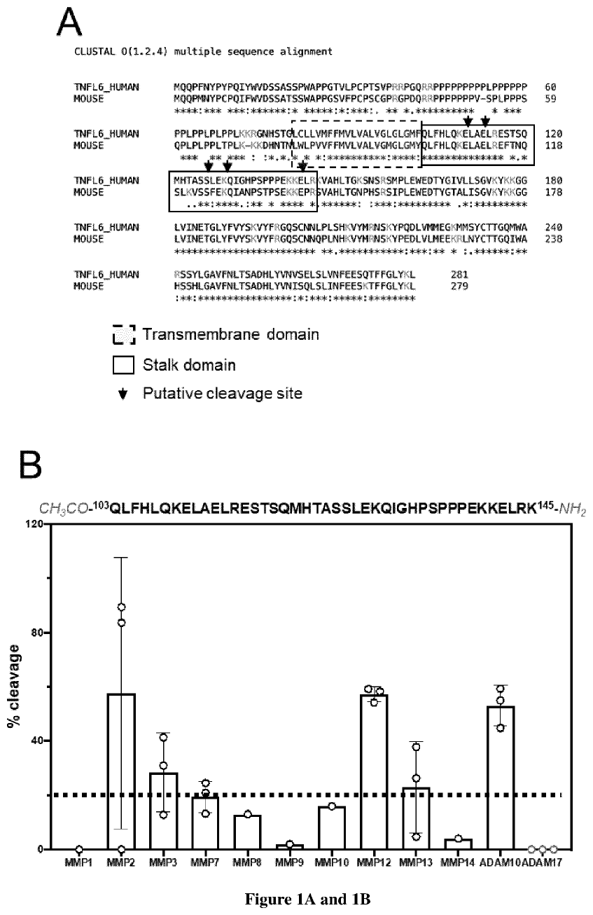

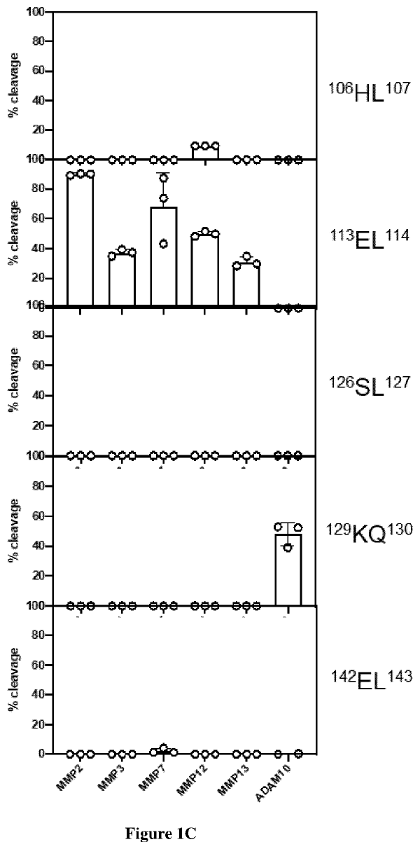

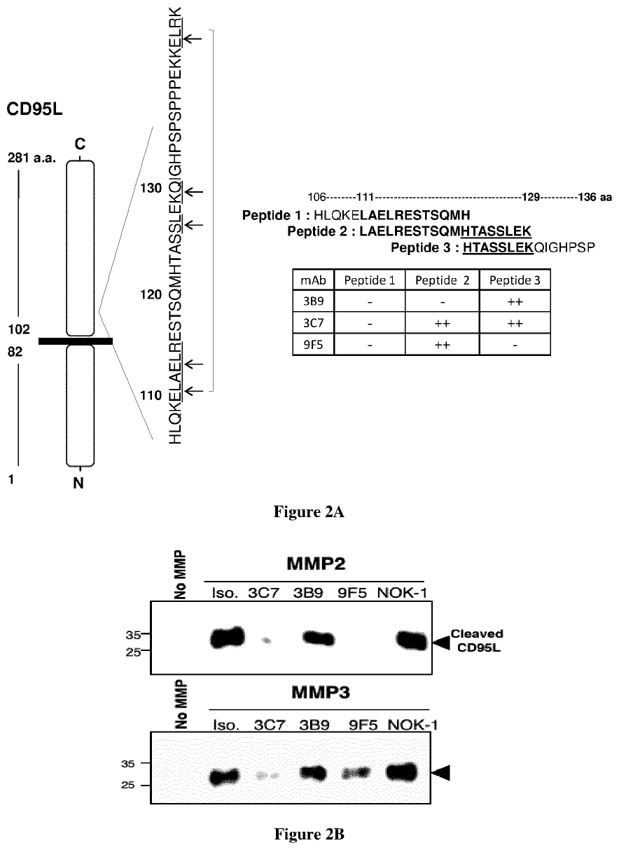

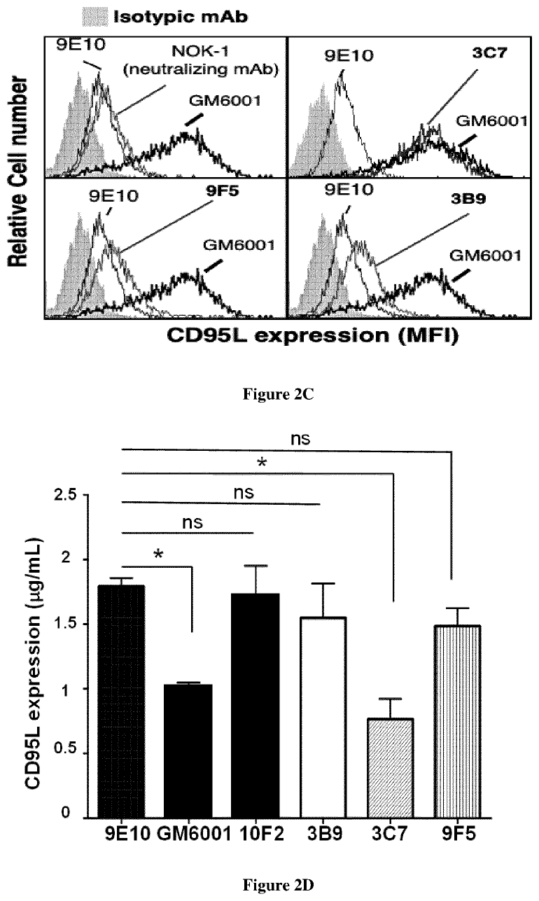

Many discrepancies exist around the way CD95L is cleaved to release the s-CD95L and up to date, different metalloprotease-cleaved sites have been suggested in CD95L sequence. We have generated different extracellular regions of CD95L fused to large domains including the Ig domain of the gp190 (LIF receptor). Their cleavage by metalloproteases engenders low-molecular sized fragments easily observable by western blotting. Using this Ig-CD95L, we showed that MMP2 and MMP3 can cleave CD95L. These proteases exhibit different cleavage sites within the stalk region of CD95L (FIGS. 1 and 2A) rendering the identification of the enzyme/s responsible for the CD95L cleavage in lupus patients difficult to address. To accurately identify the cleavage sites within CD95L and the metalloproteases responsible for this process, a 43-mer covering the stalk region of CD95L (ranging from residue 103 to residue 145) was incubated with MMPs and ADAMs at the same concentration. MMP2, MMP3, MMP7, MMP12 and MMP13 as well as ADAM10 efficiency degrades/cleaves the 43-mer (i.e more of 20% of cleavage, FIG. 1B). The privileged cleavage sites (¹¹³EL¹¹⁴ for MMPs and ¹²⁹KQ¹³⁰ for ADAM) were then determined from MS/MS sequencing of the resulting cleavage products.

We hypothesized that monoclonal antibodies targeting the Metalloprotease-Cleavage Sites of CD95L (MCSCs) could prevent the access to these enzymes and inhibit CD95L shedding. Therefore, we generated antibodies covering different epitopes within MCSC (FIG. 2A) and two of them (i.e., 3C7 and 9F5), efficiently abrogated the cleavage of CD95L by MMP2 and MMP3 in vitro (FIG. 2B, Table 1). On the contrary, the antibody 3B9 binding to the stalk region ranging from the amino acid residue 130 to 136 does not prevent the cleavage of CD95L by MMP2 and MMP3 in vitro (FIG. 2B, Table 1).

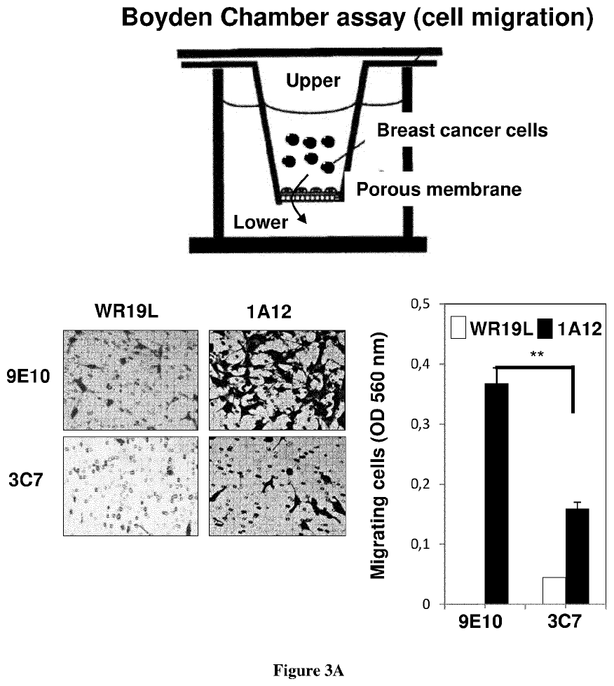

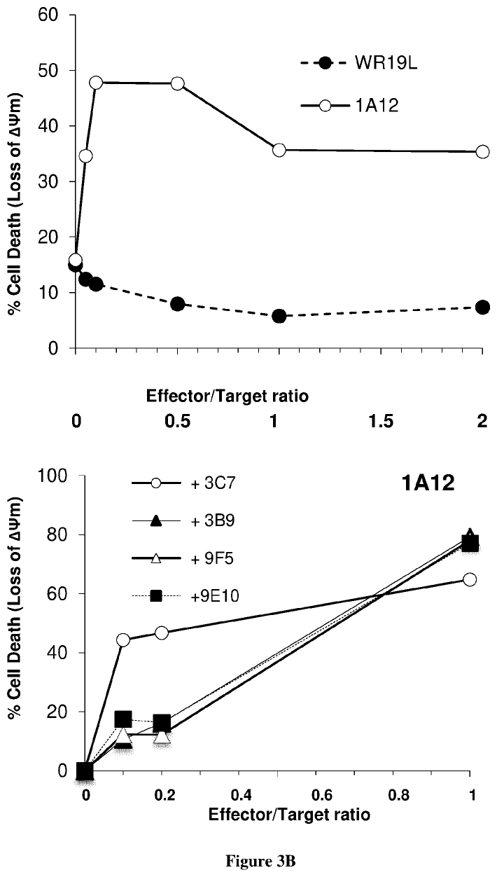

In cellulo, although incubation of CD95L-expressing T-cells (i.e., 1A12 cells) with 9F5 did not alter the expression level of membrane-bound CD95L, 3C7 treatment engendered an augmentation of m-CD95L similar to that observed with the broad spectrum MMP inhibitor GM6001 (FIG. 2C). Inhibition of CD95L cleavage led to a diminution of its soluble counterpart dosed in the supernatant (FIG. 2D). Using Boyden chamber assay, incubation of 1A12 (CD95L+) cells with 3C7 mAb inhibited the generation of s-CD95L responsible for promoting the migration of triple negative breast cancer cells MDA-MB-231 (FIG. 3A). Moreover, while 3C7 mAb blocked CD95L cleavage, it did not prevent the apoptotic function of its membrane-bound counterpart (FIG. 3B). These findings indicate that these MCSC-targeting mAbs can compete with MMPs and represent original and attractive therapeutic options to block the release of the inflammatory s-CD95L without affecting the apoptotic roles of its membrane-bound counterpart.

TABLE 1
Effect of generated antibodies on
CD95L cleavage by metalloprotease.

| | 3C7 | 9F5 | 3B9 |
|---|---|---|---|
| Inhibition of CD95L cleavage by MMP-2 in vitro | + | + | − |
| Inhibition of CD95L cleavage by MMP-3 in vitro | + | + | − |
| In cellulo inhibition of s-CD95L release (on 1A12 | + | + | + |
| cells) without alter the apoptotic function of | | | |
| membrane-bound counterpart | | | |

### REFERENCES

Throughout this application, various references describe the state of the art to which this invention pertains. The disclosures of these references are hereby incorporated by reference into the present disclosure.

1. An antibody which binds to the extracellular domain of CD95L, wherein said antibody prevents the cleavage of CD95L by at least one metalloprotease and wherein an epitope of said antibody comprises at least one amino acid residue in the amino acid sequence ranging from the amino acid residue at position 110 to the amino acid residue at position 130 in SEQ ID NO:1, wherein the antibody comprises a light chain comprising at least one or at least two of the following CDRs:
(SEQ ID NO: 2)
L-CDR1: QSLLSSR-X₈-QK-X₁₁-Y
wherein X₈ is T or N and X₁₁ is H or T
(SEQ ID NO: 3)
L-CDR2: FTS
(SEQ ID NO: 4)
L-CDR3: X₁-QHYNTPWT
wherein X₁ is K or Q,
and/or
a heavy chain comprising at least one
or at least two of the following CDRs:
(SEQ ID NO: 5)
H-CDR1: GYTFT-X₆-YW
wherein X₆ is S or T
(SEQ ID NO: 6)
H-CDR2: INP-X₄-X₅-GYT
wherein X₄ is N or R and X₅ is T or N
(SEQ ID NO: 7)
H-CDR3: AVDLGY

2. The antibody of claim 1 which prevents the cleavage of CD95L by MMP-2 and/or MMP-3.

3. The antibody of claim 1 which binds to an epitope comprising 1, 2, 3, 4, 5, 6, 7, 8, 9, 10, 11, 12, 13, 14, 15, 16, 17, 18, or 19 amino acid residues from amino acid residues 111 to 129 of SEQ ID NO: 1.

4. The antibody of claim 1 which binds to a conformational epitope.

5. The antibody of claim 1 which is a monoclonal antibody.

6. The antibody of claim 1 which is a chimeric antibody, a humanized antibody or a human antibody.

7. The antibody of claim 1 which is an antibody fragment.

8. (canceled)

9. (canceled)

10. The antibody of claim 1 which comprises a light chain comprising at least one of the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and/or a heavy chain comprising at least one of the following CDR i) the VH-CDR1 as set forth in SEQ ID NO:10 (GYTFTSYW), ii) the VH-CDR2 as set forth in SEQ ID NO:11 (INPNTGYT) and iii) the VH-CDR3 as set forth in SEQ ID NO:7 (AVDLGY).

11. The antibody of claim 1 which comprises a light chain comprising at least one of the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and/or a heavy chain comprising at least one of the following CDR i) the VH-CDR1 as set forth in SEQ ID NO:14 (GYTFTTYW), ii) the VH-CDR2 as set forth in SEQ ID NO:15 (INPRNGYT) and iii) the VH-CDR3 as set forth in SEQ ID NO:7 (AVDLGY).

12. The antibody of claim 1 which comprises a light chain comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and a heavy chain comprising the following CDR i) the VH-CDR1 as set forth in SEQ ID NO:10 (GYTFTSYW), ii) the VH-CDR2 as set forth in SEQ ID NO:11 (INPNTGYT) and iii) the VH-CDR3 as set forth in SEQ ID NO:7 (AVDLGY).

13. The antibody of claim 1 which comprises a light chain comprising the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and heavy chain comprising the following CDR i) the VH-CDR1 as set forth in SEQ ID NO:14 (GYTFTTYW), ii) the VH-CDR2 as set forth in SEQ ID NO:15 (INPRNGYT) and iii) the VH-CDR3 as set forth in SEQ ID NO:7 (AVDLGY).

14. The antibody of claim 1 which comprises a light chain having 70% of identity with SEQ ID NO: 16 or SEQ ID NO:18 and/or a heavy chain having 70% of identity with SEQ ID NO: 17 or SEQ ID NO:19.

15. The antibody of claim 1 which cross-competes for binding to the CD95L isoform with a monoclonal antibody comprising a light chain comprising the following CDR: i) the VL-CDR1 as set forth in SEQ ID NO:8 (QSLLSSRTQKHY), ii) the VL-CDR2 as set forth in SEQ ID NO:3 (FTS) and iii) the VL-CDR3 as set forth in SEQ ID NO:9 (KQHYNTPWT) and a heavy chain comprising the following CDR i) the VH-CDR1 as set forth in SEQ ID NO:10 (GYTFTSYW), ii) the VH-CDR2 as set forth in SEQ ID NO:11 (INPNTGYT) and iii) the VH-CDR3 as set forth in SEQ ID NO:7 (AVDLGY).

16. The antibody of claim 1 which cross-competes for binding to the CD95L isoform with a monoclonal antibody comprising a light chain comprising the following CDR: i) the VL-CDR1 as set forth in SEQ ID NO:12 (QSLLSSRNQKTY), ii) the VL-CDR2 as set forth in SEQ ID NO:3 (FTS) and iii) the VL-CDR3 as set forth in SEQ ID NO:13 (QQHYNTPWT) and heavy chain comprising the following CDR i) the VH-CDR1 as set forth in SEQ ID NO:14 (GYTFTTYW), ii) the VH-CDR2 as set forth in SEQ ID NO:15 (INPRNGYT) and iii) the VH-CDR3 as set forth in SEQ ID NO:7 (AVDLGY).

17. A nucleic acid molecule encoding the antibody of claim 1 or a heavy chain and/or a light chain of the antibody of claim 1.

18. A vector that comprises the nucleic acid of claim 17.

19. A host cell which has been transfected, infected or transformed by the nucleic acid of claim 18 and/or a vector encoding the nucleic acid.

20. (canceled)

21. (canceled)

22. A method of treating cancer in a patient in need thereof, comprising administering to the patient a therapeutically effective amount of the antibody of claim 1.

23. The method of claim 22 wherein the patient suffers from a cancer selected from the group consisting of breast cancer, colon cancer, lung cancer, prostate cancer, testicular cancer, brain cancer, skin cancer, rectal cancer, gastric cancer, esophageal cancer, sarcomas, tracheal cancer, head and neck cancer, pancreatic cancer, liver cancer, ovarian cancer, lymphoid cancer, cervical cancer, vulvar cancer, melanoma, mesothelioma, renal cancer, bladder cancer, thyroid cancer, bone cancers, carcinomas, sarcomas, and soft tissue cancers.

24. The method of claim 22 wherein the patient suffers from a triple negative breast cancer.

25. A method of treating autoimmune inflammatory disease in patient in need thereof, comprising administering to the patient a therapeutically effective amount of the antibody of claim 1.

26. The method of claim 25 wherein the patient suffers from systemic lupus erythematosus.

27. A pharmaceutical composition comprising the antibody of claim 1.

28. An antibody comprising means to bind to an epitope of CD95L, wherein the epitope comprises 1, 2, 3, 4, 5, 6, 7, 8, 9, 10, 11, 12, 13, 14, 15, 16, 17, 18, or 19 amino acid residues from amino acid residues 111 to 129 of SEQ ID NO: 1.


SEQUENCE LISTING (25 sequences)

SEQ ID NO: 1 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 2 - protein, 12 aa, synthetic construct
  REGION 1..12: note = L-CDR1
  VARIANT 8: note = X is T or N
  VARIANT 11: note = X is H or T
QSLLSSRXQKXY

SEQ ID NO: 3 - skipped

SEQ ID NO: 4 - protein, 9 aa, synthetic construct
  REGION 1..9: note = L-CDR3
  VARIANT 1: note = X is K or Q
XQHYNTPWT

SEQ ID NO: 5 - protein, 8 aa, synthetic construct
  REGION 1..8: note = H-CDR1
  VARIANT 6: note = X is S or T
GYTFTXYW

SEQ ID NO: 6 - protein, 8 aa, synthetic construct
  REGION 1..8: note = H-CDR2
  VARIANT 4: note = X is N or R
  VARIANT 5: note = X is T or N
INPXXGYT

SEQ ID NO: 7 - protein, 6 aa, synthetic construct
  REGION 1..6: note = H-CDR3
AVDLGY

SEQ ID NO: 8 - protein, 12 aa, synthetic construct
  REGION 1..12: note = L-CDR1
QSLLSSRTQKHY

SEQ ID NO: 9 - protein, 9 aa, synthetic construct
  REGION 1..9: note = L-CDR3
KQHYNTPWT

SEQ ID NO: 10 - protein, 8 aa, synthetic construct
  REGION 1..8: note = H-CDR1
GYTFTSYW

SEQ ID NO: 11 - protein, 8 aa, synthetic construct
  REGION 1..8: note = H-CDR2
INPNTGYT

SEQ ID NO: 12 - protein, 12 aa, synthetic construct
  REGION 1..12: note = L-CDR1
QSLLSSRNQKTY

SEQ ID NO: 13 - protein, 9 aa, synthetic construct
  REGION 1..9: note = L-CDR3
QQHYNTPWT

SEQ ID NO: 14 - protein, 8 aa, synthetic construct
  REGION 1..8: note = H-CDR1
GYTFTTYW

SEQ ID NO: 15 - protein, 8 aa, synthetic construct
  REGION 1..8: note = H-CDR2
INPRNGYT

SEQ ID NO: 16 - protein, 113 aa, Mus musculus
DIVMTQSPSSLAMSVGQKVTMSCKSSQSLLSSRTQKHYLAWYQQKPGQSPKLLVYFTSTRESGVPDRFIGSGSGTDFTLTISSVQAEDLALYYCKQHYNTPWTFGGGTKLEIK

SEQ ID NO: 17 - protein, 113 aa, Mus musculus
QVQLQQSGAELAKPGASVKMSCKTSGYTFTSYWMHWVKQRPGQGLEWIGYINPNTGYTEYIQRFRDKATLTADKSSGTAYMQLSSLTSEDSAVYYCAVDLGYWGQGTLVTVSA

SEQ ID NO: 18 - protein, 112 aa, Mus musculus
DIVMTQSPSSLAMSVGQKVTMNCKSSQSLLSSRNQKTYLAWYQQKPGQSPKLLVYFTSTRASGVPDRFIGSGGTDFTLTISSVQAEDLALYYCQQHYNTPWTFGGGTKLEIK

SEQ ID NO: 19 - protein, 113 aa, Mus musculus
QVQLQQSGAELAKPGASVKMSCKASGYTFTTYWMHWVKQRPGQDLEWIGYINPRNGYTEYNQNFKDKATLTADKSSTTAFMQLSSLTSEDSALYYCAVDLGYWGQGTLVTVSA

SEQ ID NO: 20 - protein, 42 aa, Homo sapiens
HLQKELAELRESTSQMHTASSLEKQIGHPSPSPPPEKKELRK

SEQ ID NO: 21 - protein, 17 aa, Homo sapiens
HLQKELAELRESTSQMH

SEQ ID NO: 22 - protein, 19 aa, Homo sapiens
LAELRESTSQMHTASSLEK

SEQ ID NO: 23 - protein, 15 aa, Homo sapiens
HTASSLEKQIGHPSP

SEQ ID NO: 24 - protein, 8 aa, Homo sapiens
HTASSLEK

SEQ ID NO: 25 - protein, 279 aa, Mus musculus
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSHLGAVFNLTSADHLYVNISQLSLINFEESKTFFGLYKL